(19) United States
(12) Patent Application Publication
(10) Pub. No.: US 2007/0264221 A1
(43) Pub. Date: Nov. 15, 2007

(54) Extract of a Plant Belonging to the Genus Plukenetia Volubilis and Its Cosmetic Use

(76) Inventors: Philippe Moser, Essey-Les-Nancy (FR); Olga Freis, Seichamps (FR); Veronique Gillon, Saink-Max (FR); Louis Danoux, Saulxures-Les-Nancy (FR)

Correspondence Address: COGNIS CORPORATION;PATENT DEPARTMENT, 300 BROOKSIDE AVENUE, AMBLER, PA, 19002, US

(21) Appl. No.: 11/666,769

(22) PCT Filed: Oct. 26, 2005
(86) PCT No.: PCT/EP05/11454

§ 371 Date: May 2, 2007

(30) Foreign Application Priority Data
Nov. 3, 2004 (EP) 04292611.3

(51) Int. Cl. A61K 8/97; A61K 36/47; A61Q 19/00

(52) U.S. Cl. 424/74; 424/731

(57) ABSTRACT

The invention relates to a preparation containing an extract of a plant of the family Euphorbiaceae genus Plukenetia and cosmetic and pharmaceutical preparations containing the extract. The extract contains proteins which when applied to the skin can provide an anti-inflammatory effect, a skin tightening effect, an antiaging effect and a substantivity effect. The preparations can also contain an oil of the plant. The preferred plant is Plukenetia volubilis. The extract, protein or mixture of proteins in the extract can be modified by hydrolysing, grafting or crosslinking.

The present invention relates to the cosmetic use of an extract of a plant belonging to the family Euphorbiaceae (preferably belonging to the genus Plukenetia). Furthermore it relates to the cosmetic use of a protein or a mixture of proteins, whereby said protein or said mixture of proteins is extractable from a plant belonging to the family Euphorbiaceae (preferably belonging to the genus Plukenetia). Furthermore the present invention relates to said extract or to said protein or to said mixture of proteins for use as a medicament. Furthermore the present invention relates to said extract or to said protein or to said mixture of proteins, whereby said extract or said protein or said mixture of proteins has been modified chemically or enzymatically, e.g. by crosslinking, by grafting or by hydrolysis.

FR-A 2 701 847 discloses compositions for pharmaceutical and, in particular, dermatological or cosmetic use, incorporating a protein moiety which comprises, as the active ingredient, alone or in association with at least one other active principle, albumin. The composition of the invention is characterized in that the albumin is of plant origin and is present in the composition in the form of soluble proteins in their native state, in a non-saline aqueous phase, at a pH of between 4 and 7, the soluble protein content being at least 25% by dry weight of the protein fraction. Albumin is obtained from seeds selected among leguminous, oleaginous, cereal seeds.

FR-A 2 761 264, WO 98/42305, EP-A 0 973 495 and the European patent application with the application number 01967142.9 relate to similar subjects.

It is state of the art to use vegetal proteins, e.g. albumin, in cosmetics for tightening effects.

There are documents of the state of the art that disclose a process to obtain an active principle with an immediate skin tightening effect or cosmetic compositions useful for providing a skin-tightening effect.

WO 02/28360 discloses a process for obtaining an active principle with an immediate skin tightening effect, characterized in that it comprises the following steps: dissolving in an aqueous phase a cereal press cake in the amount of 50 to 500 g/l, moderated enzymatic hydrolysis at an acid pH and a temperature comprised between 40 and 80° C., in the presence of an enzyme, separation of the soluble and insoluble phases and inactivation of the medium to interrupt the enzymatic reaction, polymerization of the retained proteins by addition of a polymerization initiator, separation of the polymerized proteins.

WO 2003/063816 discloses a topical personal care composition useful for providing a skin-tightening effect comprising at least one protein selected from the group consisting of hydrolyzed proteins, partially-hydrolyzed proteins and mixtures thereof and at least one organic powder in a dermatologically acceptable carrier; wherein the carrier is in the form of an emulsion.

EP-A 1 038 519 discloses a composition useful as a tightening agent and a method for reducing or removing the signs of cutaneous aging, comprising applying onto skin a composition comprising at least one grafted silicone polymer comprising a polysiloxane portion and a portion composed of a non-silicone organic chain, one of the two portions constituting the main chain of the polymer and the other being grafted to the said main chain.

U.S. Pat. No. 6,284,233 discloses an anti-wrinkle cosmetic or dermatological composition, comprising in a physiologically acceptable medium, a dispersion of a film-forming polymeric system comprising at least one polymer capable of forming a film permeable to water vapour, having a Young's modulus ranging from 10⁸ to 10¹⁰ N/m² and producing, after application at a concentration of 7% in water and then drying, a retraction of the isolated stratum corneum higher than 1% at a temperature of 30° C. and a relative humidity of 40%, and a dendritic polyester polymer having terminal hydroxyl functional groups.

U.S. Pat. No. 5,879,684 discloses, for tightening treatment, a skin tautening aqueous gel from the combination of water, a dispersed finely particulate vegetable based tautening or tensor agent, a polymeric gelling agent, a liquid hydrocarbon dispersing aid and a nonionic surfactant.

JP-A 100 07 518 discloses a skin lotion exhibiting a tightening effect for skin. This skin lotion contains an extract from seeds of Lupinus albus L. as a family Leguminosae plant and an extract from Thymus vulgaris L. as a family Lamiacea plant as active components. A highly polymerized protein contained in the Lupinus albus L. exhibits a tightening effect for skin and makes the surface of skin smooth by forming a film on the skin.

The problem underlying the present invention is to provide new substances useful for cosmetic and/or medical applications.

The extracts of a plant belonging to the family Euphorbiaceae, especially the extracts of a plant belonging to the genus Plukenetia, preferably the extracts of the seeds of the plant Plukenetia volubilis contain proteins with a rather low molecular weight, which in tests have shown a tightening effect on human skin. The main protein has a molecular weight close to animal albumin (60000 Da) that is a reference as tightening protein.

Some of the extracts according to the present invention show at the same protein concentration the same tightening activity as “Glycine Soja (Soybean) Protein” (INCI name) (an active ingredient purchasable from Laboratoires Serobiologiques (LS), Division de Cognis France).

Some of the extracts according to the present invention show tightening effects and substantivity effects and moisturizing effects to human skin.

Plukenetia volubilis L, also called “Inca peanut” or “Inca Inchi” or “Sacha Inche”, is a plant native to the high-altitude rain forests of the Andean region of South America. Despite its vernacular name “Inca peanut”, it does not belong to the same botanical family as the true peanut (Arachis hypogea L—family Fabaceae), but to the family Euphorbiaceae. It grows as a wine and produces a tetraglobular fruit with loculi that contains one seed with white cotyledon and a hard brown seed coat.

Although not a cultivated crop until now, the seeds collected by the wild have been a component of the diet of the Chancas Indians and other native tribal groups of the Andean regions. It is eaten either roasted, either ground and mixed with maize meal and peppers. The seeds of this plant are valued for their high oil and protein content, and the roasted seeds eaten either alone or mixed with corn meal and peppers. In Peru, the cultivation seems now to be encouraged for the extraction of the edible oil and for the remaining cake rich in proteins with good nutritional value that is used to feed the cattle.

Plukenatia volubilis oil is mixed with the flour meal by Mayorunas, Chayuhuitas, Campas, Huitotas Shipibas, Yaguas and Boras women to make a special cream to revitalize and give youth to the skin.

The seed kernels of Plukenetia volubilis are rich in oil (35 to 60%): this oil is characterized by a high content in unsaturated fatty acids (around 37% of linoleic acid and 50% of alpha-linolenic acid with high nutritional value).

The kernel has a high content in proteins (27 to 31%) that are close to that of soy bean (28%). The protein content of a defatted seed meal reaches 53% (HAMAKER B. R., VALLES C., GILMAN R., HARDMEIER R. M, CLARK D. et al. Amino Acid and Fatty Acid profiles of the Inca peanut (Plukenetia volubilis L.): Cereal Chem., 1992, volume 69(4): pages 461-463).

Only little information is available in the state of the art on the contents of Plukenetia kernels. A recent study has shown that the albumin fraction is mainly composed of a single storage protein that accounted for a substantial portion of total seed proteins (SATHE S. K., HAMAKER B. R., SZE-TAO K. W., VENKATACHALAM M., Isolation, purification, and biochemical characterization of a novel water soluble protein from Inca peanut (Plukenetia volubilis L.): J. Agric. Food Chem., 2002, volume 50(17): pages 4906-4908.

This protein was isolated by extraction followed by purification by ion exchange chromatography. The authors showed that this water soluble albumin from Inca peanut (called IPA=Inca Peanut Albumin) represented approximately 25% by weight of the weight of the defatted seed flour. IPA is a dimeric reserve protein of 3S type composed of 2 glycosylated polypeptides, with respective molecular weight of 32800 and 34800 Da. It is a basic protein (isoelectric point around 9.4) that contains all the essentials amino acids in adequate amounts when compared with the FAO/WHO recommended amino acid pattern.

The following paragraphs disclose embodiments or preferred embodiments of the present invention:
The use of the extract according to the present invention or of the proteins or of the mixture of proteins according to the present invention as active ingredient alone or in association with further active ingredients for the preparation of a cosmetic or dermocosmetic composition for topical use for skin, lips, hair or nails.
In one embodiment of the present invention the extract according to the present invention or the proteins or the mixture of proteins according to the present invention is obtainable from partially or totally defatted entire seeds or kernels (kernels are decoated seeds).
In one embodiment of the present invention the content in active ingredients of the extract according to the present invention or of the proteins or of the mixture of proteins according to the present invention can be increased by chromatography (e. g.: gel permeation chromatography, ion exchange chromatography or affinity chromatography) or by membrane separation (e. g.: ultrafiltration, dialysis or reverse osmosis) or by precipitation (solvent, salts, temperature). These techniques are examples of purification steps, i. e. according to the present invention the term “purifying” comprises, yet is not limited to, said techniques.
The extract according to the present invention or of the proteins or of the mixture of proteins according to the present invention can be modified chemically or enzymatically, e. g. by crosslinking, by grafting or by hydrolysis (enzymatic hydrolysis can be carried out by using proteases or glycosidases or lipases). These techniques are examples of modification steps, i. e. according to the present invention the term “modifying” comprises, yet is not limited to, said techniques.
The extract according to the present invention or the proteins or the mixture of proteins according to the present invention can be used to improve the general condition of the skin, lips, hair or nails.
The extract according to the present invention or the proteins or the mixture of proteins according to the present invention can be used to improve the strength of the skin, more specifically of the stratum corneum, to mechanical constraints.
The extract according to the present invention or the proteins or the mixture of proteins according to the present invention can be used to bring about anti-wrinkle or anti ageing effects, preferably to human skin.
The extract according to the present invention or the proteins or the mixture of proteins according to the present invention can be used to bring about anti-inflammatory effects, preferably to human skin.
The extract according to the present invention or the proteins or the mixture of proteins according to the present invention can be used to bring about a moisturizing effect, preferably to human skin or lips.
The extract according to the present invention or the proteins or the mixture of proteins according to the present invention can be used to bring about an anti-protease effect.
The extract according to the present invention or the proteins or the mixture of proteins according to the present invention can be used to bring about a conditioning effect to human skin, lips or hair.
In one embodiment of the present invention the proteins or the mixture of proteins according to the present invention has an average molecular weight of 60000 Da (+−5000 Da).
In one embodiment of the present invention the proteins or the mixture of proteins according to the present invention has an average molecular weight of 30000 Da (+−5000 Da).

Any treatment of the extract according to the present invention or of the proteins or of the mixture of proteins according to the present invention, may it be purification or modification, can be carried out in the presence or in the absence of the plant material (e. g. seeds) that are extracted with a solvent in order to obtain an extract.

The concentration of the proteins in the extract according to the present invention can be improved by all the methods known in the state of the art (these methods are embodiments of “purifying”), e. g.: membrane filtration technologies (e. g. ultrafiltration), chromatography (e. g.: ion exchange, affinity, adsorbtion, hydrophobic), salt precipitation, pH modification, solvent precipitation, ultrasound, use of glycosidase enzymes

The proteins and/or the extract can be used either in their native form, e. g. as described in the examples, or in modified forms (the following techniques are embodiments of “modifying”): hydrolysis either chemical either enzymatic, cross-linking (e. g. chemical or enzymatic cross-linking), modification with lipids or sugars

Properties that can be expected of many of the extracts and/or the proteins include (effects on human skin): tightening effect, anti-wrinkle effect, moisturizing effect, substantivity effect (due to the basic iso-electric point) and stimulation of the skin cell metabolism for a final anti-aging effect

The association between an extract according to the present invention and oil of the same plant is of interest for a cosmetic and/or dermatologic product such as a double serum for moisturizing, nutrition, regenerating activities.

### EXAMPLES

% means % by weight.

### I: Preparation of Extracts of Plukenetia volubilis seeds

### Example 1

The raw material is constituted by entire seeds of Plukenetia volubilis. The brown seed coats are hand removed to obtain the white kernels. The kernels are then milled and the flour is extracted with hexane to obtain defatted kernel flour.
In a reactor 100 g of defatted kernel flour cakes are introduced
1 litter of distilled water is added
The pH of the solution is adjusted to 7.0 under shaking with NaOH 4N
Extraction is carried out under shaking for 2 hours at room temperature at pH 7.0
Centrifugation is carried out for 15 min at 5000 g
The supernatant is collected, then filtered up to 0.45 μm to obtain a white extract
This extract is finally freeze-dried (or could be spray-dried).

### Example 2

The raw material is different compared to the one of example 1 and is constituted by the cake resulting from the cold extraction (by press) of Plukenetia volubilis oil from kernels (seed coats removed). The cake is defatted with hexane to remove the remaining oil.
In a reactor 200 g of defatted cake flour are introduced
Add 2 liters of distilled water
The extraction is then performed as in example 1.

Characteristics of the 2 extracts are listed in the following table

| | According to | According to |
|---|---|---|
| raw material | example 2 | example 1 |
| Content of defatted meal | 55% | 56.3% |
| dried extract of the final solution | 2.91% | 3.39% |
| weight of lyophilisate obtained | 44.2 g | 20.8 g |
| Yield Extract/defatted meal (on dry weight | 22.1% | 20.8% |
| basis) | | |
| Protein content of the lyophilisate | 62.7% | 66.9% |
| (Nitrogen × 6.25) | | |
| Yield of extracted proteins/proteins in the | 25.7% | 20.8% |
| defatted meal | | |

Molecular weight of the proteins present in the extract was determined by gel electrophoresis.

SDS page in non reducing conditions shows 2 main protein bands corresponding to a molecular weight of approximately 60 000 Da and 30 000 Da.

If the electrophoresis is performed in reducing conditions (cleavage of disulfide bonds by mercaptoethanol it remains only the band at 30 000 Da). This means that the band at 60,000 Da is a dimeric protein constituted by 2 sub-units of approximately 30 000 Da linked by disulfide bonds. Isoelectrofocalisation performed on an extract enriched by ion exchange in 60 000 Da protein has also shown that its isoelectric point of this protein basic (near 9): this band certainly corresponds to the IPA described in the state of the art.

### II: Tightening Effect Ex-vivo

Principle: The principle is to measure skin displacement in response to a small sinusoidal force applied parallel to a skin surface (a sample of human skin). The explored parameter is the dynamic spring rate (DSR) (ratio force/displacement). The DSR is the ratio of force divided by the displacement. The units of force and displacement are not important in this case, as the evaluation is always done in the relative way. Said ratio is calculated continuously each minute and the results are expressed as the percentage of DSR modification referring to time 0 (=before treatment). The units may be g/mm or N/m etc. Said ratio is calculated on the basis of electrical signals and the force and displacement are in mV. (No importance of units because the evaluation is done in the relative way and the DSR is calculated in %!) Softening of the skin due to treatment with an appropriate active ingredient is expressed by diminution of DSR while tightening of the skin or firming is reflected by an increase in DSR.

Details of the method: The skin was mounted on a glass microscope slide. The mounted skin was equilibrated during 2 hours in an atmosphere with controlled humidity (relative humidity, RH) and temperature T (T=20° C., RH=33%). The mechanical properties were tested under 5 conditions:
1. Control without treatment
2. Skin treated with placebo: Sepigel 1% (Sepigel 305, purchasable from the company Seppic: polyacrylamide (and) isoparaffin (and) laureth-7)
3. Skin treated with Plukenetia extract according to example 2 at 0.75% by weight in the placebo: that mean on the basis of the protein content 4.7 g/l of proteins
4. Skin treated with Plukenetia extract according to example 2 at 1.5% by weight in the placebo that mean on the basis of the protein content 9.4 g/l of proteins
5. Skin treated with the Cognis active ingredient of INCI name: glycine soja (soybean) protein diluted at 10% in the placebo, that means on the basis of the protein content 4.7 to 5 g/l of proteins.

The results are expressed as the evolution of the DSR parameter after 180 minutes.

TABLE 1
tightening activity of Plukenetia extract at
0.75% in a placebo formulation

| | | SEM (Standard | |
|---|---|---|---|
| DSR value after 180 min (%) | Average | Error Mean) | Statistics |
| Control | 108.71 | 3.18 | |
| Placebo | 125.53 | 4.27 | |
| Plukenetia extract according to | 179.8 | 14.2 | ** |
| example 2 at 0.75% (w/v) | | | |
| in placebo formulation | | | |
| Cognis (= LS) active ingredient of | 179.66 | 16.94 | ** |
| INCI name: glycine soja (soybean) | | | |
| protein. | | | |
| at 10% in placebo formulation | | | |

| % increase of DSR referring to placebo | | |
|---|---|---|
| at 180 min (%) | Average | SEM |
| Plukenetia extract according to example 2 | 43.54 | 11.20 |
| at 0.75% (w/v) in placebo formulation | | |
| LS active ingredient of INCI name: glycine | 42.22 | 9.40 |
| soja (soybean) protein at | | |
| 10% in placebo formulation | | |

TABLE
tightening activity of Plukenetia extract
at 1.5% in placebo formulation

| DSR value after 180 min (%) | Average | SEM | Statistics |
|---|---|---|---|
| Control | 104.18 | 0.56 | |
| Placebo | 134.41 | 11.71 | |
| Plukenetia extract according to example 2 | 262.16 | 43.47 | *** |
| at 1.5% (w/v) in placebo formulation | | | |
| LS active ingredient of INCI name: | 187.13 | 20.44 | ** |
| Glycine Soja (Soybean) Protein. | | | |
| at 10% in placebo formulation | | | |

| % increase of DSR referring to placebo at | | |
|---|---|---|
| 180 min (%) | Average | SEM |
| Plukenetia extract according to example 2 | 91.00 | 19.11 |
| at 1.5% (w/v) in placebo formulation | | |
| LS active ingredient of INCI name: | 39.18 | 11.18 |
| Glycine Soja (Soybean) Protein at | | |
| 10% in placebo formulation | | |

**: significant referring to the placebo and control.
***: significant referring to the placebo and control and to Plukenetia extract at 0.75%.

The tightening effect of the Plukenetia extract at 0.75% (˜5 g/l of proteins) was significant referring to the placebo and control and as effective as LS active ingredient of INCI name: Glycine Soja (Soybean) Protein at 5 g/l proteins: a dose-effect activity is observed with Plukenetia extract at 1.5%.

### III: Tightening Effect In-vivo

The tightening effect in vivo was evaluated with a corneospinometer. This device allows the evaluation of the behaviour of the stratum corneum under microtorsion. Angular skin deformation was measured according to a mechanical torque applied to the skin surface by a probe, which comprised a small needle (diameter of the needle 0.8 millimeters). The contact area was lower than 0.2 millimeters and the appraisal was limited to the stratum corneum layer. A special technology allowed to lower the needle till the skin was contacted in the axis of the probe, so that only the weight of the needle was applied on the skin and parasitic friction was decreased. The device comprised a coil, which had two functions. Firstly, it applied a periodic torque to the needle. Secondly, it measured the angular positioning of the needle.

The result is expressed by a Dynamic Spring Rate (DSR), which is the ratio between mechanical torque (constant sinusoidal amplitude) and angular skin deformation (variable).

An increase of DSR corresponds to a tightening effect on the stratum corneum.

Method/test conditions:

1 volunteer
   forearm internal face
   area: 3×3 cm²
   applied dose was 3 mg/cm²
   average of 50 measurements
   results are expressed as % of increase of DSR at 10 minutes referring to T0
   Skin was treated with:
   placebo: Emulgade® CM at 20% in water: INCI name: Cetearyl Isononanoate (and) Ceteareth-20 (and) Cetearyl Alcohol (and) Glyceryl Stearate (and) Glycerin (and) Ceteareth-12 (and) Cetyl Palmitate
   LS active ingredient of INCI name: Glycine Soja (Soybean) Protein at a concentration of 10% by weight in Emulgade® CM (4.5-5 g/l of proteins)

Plukenetia extracts according to examples 1 and 2 were solubilized in water at a concentration of 100 g/l (w/v) and the solution is filtered at 0.8 μm.

The solution is then diluted in the Emulgade® CM to obtain a final extract concentration of 0.8% (w/v) that means 4.7-5.4 g/l of proteins.

| | % increase of DSR (T10/T0) |
|---|---|
| Product tested | Essay 1 |
| Placebo (Emulgade ® CM) | −18.31% |
| LS active ingredient of INCI name: | 25.99% |
| Glycine Soja (Soybean) Protein (10% in | |
| Emulgade ® CM) | |
| Plukenetia extract according to example 2 | 16.91% |
| at 0.8% (w/v) in Emulgade ® CM | |
| Plukenetia extract according to example 1 | 29.75% |
| at 0.8% (w/v) in Emulgade ® CM | |

Both extracts of Plukenetia volubilis at 0.8% (w/v) i.e. 4.5-5.4 g/l of proteins in Emulgade® CM at 20% in water have shown an immediate tightening effect as demonstrated by the increase of DSR at 10 minutes compared to T0. At the same protein concentration range, Plukenetia extracts showed approximately the same tightening effect than LS active ingredient of INCI name: Glycine Soja (Soybean) Protein.

### IV: Preparation of Hydrolysates From Plukenetia volubilis Seeds With Subtilisin A

### Example 3

In this example the enzyme used is subtilisin a marketed by the company NOVO (Alcalase 2.4 L). The starting material is a cake obtained after cold press extraction of the oil from Plukenetia volubilis kernel (remaining oil 13.5%). The cake is milled to obtain a cake flour. The experiment has been carried out in the following way:
In a reactor 75 g of cake flour are introduced
Add 750 ml of distilled water
After homogenization, adjust under shaking the pH of the mixture to 8.2 with sodium hydroxide
Heat the mixture up to 55° C.
Add to the mixture 1.9 ml of enzyme (5% based on protein content of the cake flour)
Hydrolysation for 2 hours at pH=8.0-8.2 and 55° C.
Adjust the pH to 7.0 and inactivate the enzyme by heating the mixture 10 minutes at 90° C.
Cool to room temperature and centrifuge at 5000 g
Collect the supernatant, then filter up to 0.45 micron
The hydrolysate is finally spray-dried to give 17 grams of powder (yield of spray-drying=80%) with a protein content of 66% (based on nitrogen determination)

### V: Preparation of Hydrolysates From Plukenetia volubilis Seeds With Protease From Papaya

In this serial of trials, the enzyme used has been a mixture of proteases from papaya marketed by the company BIOZYM.

### Example 4

The starting material is the same as in example 1. The experiment has been carried out in the following way:
In a reactor 75 g of cake flour are introduced
Add 750 ml of distilled water
After homogenization, adjust under shaking the pH of the mixture to 7.5 with sodium hydroxide
Heat the mixture up to 50° C.
Add to the mixture 6 grams of enzyme powder (15% based on protein content of the cake flour)
Hydrolysation for 2 hours at pH 7.5 and 50° C.
Inactivate the enzymes by heating the mixture 10 minutes at 90° C.
Cool to room temperature and centrifuge at 5000 g
Collect the supernatant, then filter up to 0.22 micron
The hydrolysate is finally spray-dried to give 13 grams of powder (yield of spray-drying=62%) with a protein content of 61%

### Example 5

The starting material is a cake obtained after cold press extraction of the oil from Plukenetia volubilis kernel and further defatted by hexane (remaining oil <0.3%) to obtain a defatted kernel flour. The experiment has been carried out in the following way:
In a reactor 40 g of defatted kernel flour are introduced
Add 360 ml of distilled water
After homogenization, adjust under shaking the pH of the mixture to 5.5 with sulfuric acid
Heat the mixture up to 50° C.
Add to the mixture 2.18 grams of enzyme powder (8% based on protein content of defatted kernel flour)
Hydrolysate during 4 hours at pH 5.5 and 50° C.
Inactivate the enzyme by heating the mixture 10 minutes at 90° C.
Cool to room temperature
Centrifuge at 5000 g, then collect the supernatant and filter up to 0.22 micron.
The hydrolysate is finally freeze-dried to give 6.5 grams of powder with a protein content based on nitrogen determination of 50%

### VI: Regenerative and Revitalizing Effects on Human Fibroblasts in Culture

2. Aim of the tests carried out:
The purpose of these tests was to evaluate the revitalizing and regenerating activities of the extracts on human fibroblasts cultured in vitro.

3. Method used:
The growth efficacy test was carried out on human fibroblasts in order to evaluate the regenerating and the growth actor like activities. The survival efficacy test was carried out on human fibroblasts, to evaluate the regenerating and the revitalizing activities. The cell number and the cell viability have been determined by recording the following parameters:
The DNA levels were evaluated with a fluorescent probe so called Hoechst 33258 (DESAULNIERS D., LEINGARTNER K., ZACHAREWSKI T. and FOSTER W. G.: Optimisation of an MCF7-E3 cell proliferation assay and effects of environmental pollutants and industrial chemicals: Toxic. In vitro, 1998, volume 12(4): pages 409-422).
ATP or adenosine triphosphate is a compound rich in energy and mainly produced from mitochondria. The cells need ATP for the activity of many enzymes which control the cytoskeleton, the ionic channels, the nutriment intake, and a lot of other biological processes (VASSEUR P., AERTS C.: Appréciation de la cytotoxicité par la mesure de l'ATP, J. Français Hydrol., 1981, volume 9, pages 149-156).
The protein levels were evaluated according to the Bradford's method (BRADFORD M. M. a rapid and sensitive method for the quantitation of microgram quantities of protein utilizing the principle of protein-dye binding: Anal. Biochem, 1976, volume 72: pages 248-254).
Glutathione (GSH) is a peptide produced by the cells to protect them from oxidative stress or certain pollutants like mercury or lead. The three aminoacids involved into the reduced form of GSH are linked by specific cytoplasmic enzymes which use ATP. The GSH was evaluated by the method of Hissin (HISSIN P. J., HILF R. A fluorometric method for determination of oxidized and reduced glutathione in tissues: Anal. Biochem., 1976, volume 74: pages 214-226).

The results were calculated referring to standard range and then in percentage versus the control (cell culture medium without products) and finally expressed as a mean from typically 1 or 2 assays in triplicate.

Results (%/control)

TABLE 1a
regenerating effect of hydrolyzed Plukenetia volubilis extracts

| | Enzyme used to | Dose | |
|---|---|---|---|
| | hydrolyze the proteins | (%, w/v) | Proteins |
| Control | / | / | 100 |
| Extract prepared according | papaine | 0.1 | 167 |
| to example 4 | | | |

These data (table 1a) show that papaine hydrolysed plukenetia volubilis extract improved cells growth and presented a good potential to simulate skin renewal and delay skin aging.

TABLE 1b
regenerating and revitalizing effect of hydrolyzed
Plukenetia volubilis extracts

| | Enzyme | | | | | |
|---|---|---|---|---|---|---|
| | used to | | | | | |
| | hydrolyze | | | | | |
| | the proteins | | | | | |
| | extract | | | | | |
| | during it | Dose | | | | GSH/ |
| | preparation | (%, w/v) | DNA | ATP | Prot. | Prot. |
| Control | | / | 100 | 100 | 100 | 100 |
| Extract prepared | alcalase | 0.03 | 105 | 127 | 133 | 113 |
| according to | | 0.1 | 120 | 144 | 157 | 123 |
| example 3 | | | | | | |
| Extract prepared | papaine | 0.03 | 112 | 132 | 129 | 125 |
| according to | | 0.1 | 129 | 182 | 162 | 145 |
| example 4 | | | | | | |
| Extract prepared | papaine | 0.03 | 105 | 141 | 128 | 124 |
| according to | | 0.1 | 129 | 161 | 153 | 115 |
| example 5 | | | | | | |

These data (table 1b) show that hydrolysed plukenetia volubilis extracts stimulated cellular protein synthesis and release of energy. Moreover, these extracts increased the rate of glutathion and therefore enhanced the cell capacities to fight against oxidative stress and pollutants.

Hydrolyzed Plukenetia volubilis extracts constitute good candidates to simulate the metabolism of skin cells and rejuvenate the aged skin. Furthermore, they improve the skin cell capacities to cope with oxidative stress and pollutants.

### VII: Substantivity Effect on Hair

4. Aim of the Tests Carried Out:

The purpose of this test was to evaluate the substantive effect on human hair damaged by bleaching and permanent wave. The substantivity of a product can be defined as the ability to be adsorbed and to adhere on keratin and to resist to a water rinse. The quantity of substantive product being adsorbed on hair is evaluated by the reaction with fluorescamine (presence of primary amines) after desorption in 2 different conditions: high temperature (50° C.) and high ionic strength (0.5M NaCl). (MINTZ G R, REINHART G M, LENT B: Relationship between collagen hydrolyzate molecular weight and peptide substantivity to hair. J. Soc. Cosmet. Chem. 1991, volume 42: pages 35-44).

5. Method used:

6. A: Preparation of the damaged hair locks (bleached and permed)

12 hair locks (1 g) were put in contact 30 mn with 5% hydrogen peroxide with a neutral pH at 30° C. and then carefully washed. Thereafter the tresses were immersed in the reducing agent (sodium thioglycolate at 5%) for 20 mn, water rinsed and followed by an immersion in neutralizing solution (3% hydrogen peroxide) for 10 mn. Finally, bleached and permed hair were washed with the 5% sodium lauryl sulfate solution, then rinsed and dried up in the oven at 45° C.

B: Absorption and removal of proteinsipeptides from the hair tresses

Absorption: Each lock was soaked either in the solution of Plukenetia volubilis extracts (0.75%) or in distilled water (10 ml) at 25° C. The locks were then washed with warm running water in order to remove not specifically bound proteins.

Desorption of the adsorbed extract on hair: The desorption of bound proteins/peptides was carried out in two successive steps:
1. each lock was placed in 10 ml of distilled water in the water-bath at 50° C. for 2 hours (=heat soak)
2. each lock was then soaked for 16 hours in 10 ml of a NaCl solution (0.5 M) at 25° C. (=high salt soak)

C: Dosage of peptides

The dosage of the desorbed protein/peptides was done in the extraction liquids (heat and high salt soak). The presence of NH2 groups was determined after the reaction with fluorescamine. The relative fluorescence was measured. The excitation and emission wavelengths were set at 390 nm 480 nm, respectively. The desorbed quantity of Plukenetia volubilis extracts from hair was determined according to the standard calibration done in the same conditions.

Results

TABLE 2
Total desorption of proteins/peptides (i.e. heat and high
salt soak) due to the treatment with Plukenetia volubilis
extracts or with distilled water (control) from bleached and
permed hair (mg of protein/g of hair)

| | Concentration of | Concentration | Total desorption |
|---|---|---|---|
| | the extract in | in proteins | (mg of protein/g |
| | % (w/v) | in g/l | per hair) |
| Control | 0 | 0 | 9.8 ± 0.5 |
| non treated hair | | | |
| Hair treated with | 0.75 | 5 | 17.8 ± 0.4 (**) |
| extract prepared | | | |
| according to | | | |
| example 1 | | | |
| Hair treated with | 0.8 | 5 | 19.5 ± 0.5 (**) |
| extract prepared | | | |
| according to | | | |
| example 2 | | | |

Statistics:
Average of 10 measures ± SEM
1 factor ANOVA; PLSD of Fisher
**: p < 0.0001 (significant/control hair)

The substantivity of Plukenetia volibilis extracts is characterized by the enhanced desorption of proteic/peptidic material form the treated hair (17.8±0.4 mg/g hair and 19.5±0.5 mg/g hair in comparison to 9.8±0.5 mg/g hair). This increase represents +87% for the extract prepared according to example 1 and +105% for extract prepared according to example 2.

1. A cosmetic use of an extract of a plant belonging to the family Euphorbiaceae (preferably belonging to the genus Plukenetia).

2. A cosmetic use of a protein or a mixture of proteins, whereby said protein or said mixture of proteins is extractable from a plant belonging to the family Euphorbiaceae (preferably belonging to the genus Plukenetia).

3. The cosmetic use according to claim 1 or 2 for the cosmetic treatment of human skin or human hair.

4. The cosmetic use according to claim 3 for tightening human skin or for bringing about an anti-ageing effect to human skin or for bringing about a substantivity effect on human hair or human skin.

5. The cosmetic use according to any of claims 1 to 4, whereby the plant is the plant Plukenetia volubilis.

6. The cosmetic use according to claims 1, 3, 4 or 5, whereby the extract is an extract of the seeds of said plant or according to claim 2, whereby said protein or said mixture of proteins is extractable from the seeds of said plant.

7. The cosmetic use according to claims 1, 3, 4 or 5, whereby the extract is an extract of the kernels of the seeds (preferably of the defatted kernels of the seeds) of said plant or according to claim 2, whereby said protein or said mixture of proteins is extractable from the kernels of the seeds (preferably of the defatted kernels of the seeds) of said plant.

8. The cosmetic use according to any of claims 1 or 3 to 7, whereby the extract is obtainable by a process comprising
a) extracting said plant or said seeds or said kernels with a solvent selected from the group consisting of alcohols with 1 to 6 carbon atoms, esters of carboxylic acids with 1 to 6 carbon atoms and alcohols with 1 to 6 carbon atoms, ketones with 1 to 6 carbon atoms, hexane, glycols with 2 to 6 carbon atoms, water and mixtures thereof to obtain a mixture comprising the extract and the solvent and
b) Removing the solvent from the mixture thus obtained or according to claim 2 whereby said protein or said mixture of proteins is extractable by a process comprising a) and b).

9. The cosmetic use according to claim 8, whereby the process further comprises
c) Treating the extract or the protein or the mixture of proteins, either before removing the solvent or after removing the solvent, whereby this treating is selected from the group consisting of purifying and modifying.

10. The cosmetic use according to claim 8 or 9, whereby the solvent is water.

11. The cosmetic use according to any of claims 1 to 10, whereby the extract or the protein or the mixture of proteins is used together with or in combination with an oil of the plant (preferably an oil of the seeds of the plant, more preferably an oil of the kernels of the seeds of the plant) according to the present invention.

12. The extract or the protein or the mixture of proteins as defined in any of claims 1 to 11 for use as a medicament.

13. The use of the extract or the protein or the mixture of proteins as defined in any of claims 1 to 11 for the manufacture of a medicament for the treatment of inflammations.

14. The extract or the protein or the mixture of proteins obtainable by the process of claim 9, whereby treating is modifying.
